(19) United States
(12) Patent Application Publication
(10) Pub. No.: US 2025/0325181 A1
(43) Pub. Date: Oct. 23, 2025

(54) Device for Examining the Anterior Part of the Eye by Retro-Illumination of Said Anterior Part of the Eye

(71) Applicants: UNIVERSITE JEAN MONNET SAINT ETIENNE, SAINT ETIENNE (FR); UNIVERSITE PARIS-SACLAY, GIF-SUR-YVETTE (FR); CENTRE HOSPITALIER UNIVERSITAIRE DE SAINT ETIENNE, SAINT ETIENNE (FR); CENTRE NATIONAL DE LA RECHERCHE SCIENTIFIQUE (CNRS), PARIS (FR); INSTITUT D'OPTIQUE, PALAISEAU (FR)

(72) Inventors: Philippe GAIN, Lyon (FR); Anthony AIN, SAINT ETIENNE (FR); Gilles THURET, SAINT BONNET LES OULES (FR); Thierry LEPINE, SAINT-HEAND (FR)

(21) Appl. No.: 18/870,718

(22) PCT Filed: Jun. 1, 2023
(86) PCT No.: PCT/FR2023/050769

§ 371 Date: Dec. 2, 2024

(30) Foreign Application Priority Data
Jun. 3, 2023 (FR) FR2205394

(51) Int. Cl. A61B 3/15

(52) U.S. Cl. CPC A61B 3/156

(57) ABSTRACT

The invention relates to a device for examining the anterior part of the eye by retro-illumination of said anterior part. This device has means for illuminating the eye, comprising a light source (1), and means for imaging the anterior part of the eye. The invention consists in the device further comprising means of modulating at least one region of the light beam emitted from the light source (1).

### TECHNICAL DOMAIN AND TECHNOLOGICAL BACKGROUND

The invention relates to the field of ophthalmology. More particularly, the invention concerns a device for carrying out an examination of the eye, more precisely of the anterior part of the eye comprising the anterior segment of the eye, by retroillumination, which consists of observing the tissues (cornea, iris, crystalline lens) illuminated by the light backscattered by the pigmented epithelium under the retina (which generates the red reflection in the pupil in photos taken with flash), the light originating from a light projected onto the eye from a light source. This type of device provides access to imaging of the anterior segment of the eye.

In the present application, anterior part of the eye refers to both the area that goes from the front face of the cornea to the rear face of the crystalline lens (commonly called anterior segment), and the vitreous body located behind the crystalline lens and upstream of the retina. Indeed, by retroillumination, it is possible to obtain an image of floating bodies located behind the crystalline lens, in the vitreous body. It can also be considered that the anterior part of the eye examined corresponds to the outer tunic of the eye (area from the front of the cornea to the back of the crystalline lens) and the middle tunic (vitreous body).

Ophthalmologists commonly use retroillumination in two types of situations.

Firstly, retroillumination is used during an examination performed with a biomicroscope, also called a slit lamp, which makes it possible to observe the anterior segment of the eye directly. The biomicroscope (or slit lamp) is a double (binocular) microscope whose light source can be moved and which allows the examination of the eyes, by inspecting the ocular surface, i.e., the conjunctiva and the cornea, as well as the internal structures of the eye such as the crystalline lens and the retina at the fundus. The human eye is an easily accessible organ, but its size (23 mm in diameter) makes it difficult to study accurately with the naked eye. Some details of the cornea, iris, crystalline lens are around a few tens of microns. In ophthalmology, the anterior segment of the eye is mainly examined to detect any abnormalities of the ocular surface.

When using a slit lamp, because of the light projected on the patient's eye, there are constantly reflections on the cornea, and sometimes on the crystalline lens as well. However, during the examination, the practitioner manually moves the light source; this movement makes it possible to move the troublesome reflection aside to thus be able to observe the entire ocular surface. But if this slit lamp is used for taking a photo, the presence of reflections makes it impossible to observe the entire cornea or crystalline lens in a single image.

However, a digital and well-resolved image of the cornea, iris or crystalline lens in retroillumination and without reflections is essential to analyze images to quantify the stage of certain pathologies.

The article by Timothy D. Weber and Jerome Mertz “In vivo corneal and lenticular microscopy with asymmetric fundus retroillumination” describes a retroillumination process in which, in order to improve the phase-gradient contrast, asymmetric illumination is used by illuminating one side of the fundus and imaging with a microscope lens, allowing images to be obtained with a lateral resolution of around one micrometer according to a field of view of 1 mm diagonal in the central cornea.

While this technique allows for well-resolved images, it is restricted to the central cornea and does not allow imaging of the anterior segment of the eye, which is much larger than the opening offered by the central cornea alone along a diagonal of 1 mm, rather it extends for several millimeters depending on the pupil opening. However, it can be particularly advantageous to image the anterior segment of the eye, including areas of the cornea that are eccentric with respect to the central cornea. It has actually been discovered that early stages of certain diseases, and especially forms of Fuchs' dystrophy, result in defects appearing in an eccentric area of the cornea, for example in front of the iris-pupil interface, before migrating to the central part. A method that only allows imaging of the central cornea cannot detect these early stages. The method proposed by this article, requiring the imaged area to be surrounded by a non-imaged light ring, does not allow imaging eccentric areas of the cornea, because the iris is an obstacle to illumination.

The second type of use occurs with an operating microscope, for cataract surgery. This surgery is the leading surgery in the world and represents 25 to 30 million procedures per year, including about 1 million per year in France. The red reflection obtained by retroillumination makes it possible to see the crystalline lens to be operated on very well. However, the reflections of the light source on the cornea are troublesome because they dazzle the operator and mask certain areas.

In order to eliminate parasitic reflections, EP-A-3336597 proposes a device of the type comprising a light source for illumination, an image sensor and two complementary polarization subas-semblies for reducing or eliminating reflections. The first polarization subassembly polarizes the illumination light directed at the observed object. In specular reflection, the polarization of the illumination light is maintained and thus the light reflected from the surface of the eye is filtered by the second polarization subassembly present in the observation path. The illumination light that is refracted to the retina is backscattered and depolarized by the retina. In addition, the light reflected by the cornea retains its polarization. However, since the cornea is composed of five layers, at each interface a part of the light undergoes reflection. The more layers the light passes through, the more its polarization will change slightly compared to the initial state, creating a diffuse reflection. Consequently, in the observed light, the second polarization subassembly will filter the reflected light having the initial polarization state and retain only the backscattered light which is depolarized. A large part of the specular reflections is thus eliminated. However, in the observed light, a portion is gener-ated by diffuse reflection and has a random polarization, thereby allowing it to pass at least partially through the second polarization subassembly. Such a device therefore makes it possible to attenuate specular reflections but does not eliminate them completely, in particular those related to diffuse reflection. Moreover, as the illumination and observation paths are merged and provide a beam splitter, light intensity for observation is lost. To compensate, a polarizing beam splitter may be provided. Such a device is therefore complex to produce and expensive and, furthermore, does not eliminate all reflections.

### PRESENTATION OF THE INVENTION

The main object of the present invention is therefore to propose a device allowing an examination and especially taking an image of the anterior segment with a less complex structure while making it possible to obtain an image in which at least the parasitic reflections intrinsic to the system are eliminated.

The object of the present invention is to propose a device for examining the anterior part of the eye by retroillumination of said anterior part of the eye, comprising means for illuminating the eye having a light source and configured to emit a light beam, and means for imaging the anterior part of the eye comprising an optical sensor configured to capture an image of the anterior segment of the eye, further comprising modulation means configured to extinguish at least one region of the light beam emitted from the light source and thus to create at least one non-illuminated region (ZNI) on the surface of the eye.

The modulation means may comprise a shutter placed in the light beam to block rays therefrom, thus extinguishing a region of the light beam. The modulation means may also be

Advantageously, the modulation means of one or more parts of the light beam sent to the patient's eye makes it possible to illuminate the eye in a modular manner.

Preferably, this illumination of the eye can be selective, especially avoiding the direct illumination of one or more regions of the eye responsible for conventional specular reflections. The modulation means thus makes it possible to create so-called “extinguished” non-illuminated regions on the surface of the eye to be examined, the modulation consisting of suppressing one or more parts of the light beam, which form these extinguished regions thus no longer generating reflections when the image is taken. This suppression of one or more parts of the light beam may be effected by shutter means interposed on the passage or by non-reflection means of one or more parts of the beam.

The regions responsible for conventional specular reflections are therefore no longer illuminated directly by the light source of the device according to the invention but rather only by the backscattering of the light by the retina, which makes the image homogeneous and without reflections.

Preferably, the imaging means is configured so that the image sensor comprises in its field of view the non-illuminated region and at least one region illuminated by the light beam on the surface of the eye. Only the regions responsible for specular reflections are not illuminated, which makes it possible to illuminate the other regions, introducing enough light into the eye for good backscattering. In return, all regions, whether illuminated by the light beam or not, are in the field of view, in order to maximize the extent of the tissues imaged in the anterior segment of the eye, and in particular for the eccentric parts of the cornea.

Typically, the imaging means is configured to present a field of view covering an area with a diameter (largest dimension) greater than or equal to 4 mm at the surface of the eye, and more preferably greater than 5 mm. Preferably, a non-illuminated region extends over an area with a diameter (largest dimension) less than or equal to 3 mm, and preferably less than or equal to 2 mm.

Preferably, the modulation means is configured so that an extinguished region is surrounded by a non-extinguished region in the light beam. Thus, a region not illuminated by the light beam on the surface of the eye is surrounded by a region illuminated by the light beam on the surface of the eye, thus limiting the inhomogeneity of the illumination by backscattering. More preferably, an extinguished region is at the center of the light beam, and the non-illuminated region is at the center of the cornea.

The imaging means comprises an image capture apparatus. Preferably, the imaging means comprises an optical unit, polarizing means and an image capture apparatus. The image of the eye can thus be captured on an optical sensor that photographs the imaging plane at the eye. In the imaging means, polarizing means such as an analyzer (polarizer) are provided which extinguish the secondary reflections related to the part of the projected light beam reflected by the cornea and the crystalline lens. The light beam composed of the refracted part in the eye to the retina is backscattered and depolarized by the retina, which makes it possible to illuminate and observe the tissues (cornea, iris, crystalline lens) illuminated by this backscattered light by the pigmented epithelium under the retina, the extinguished regions thus being illuminated by said backscattered light. The analyzer can then polarize the light beam emitted by the eye while extinguishing, i.e. suppressing, parasitic reflections intrinsic to the system and possible small secondary reflections of the corneal surface.

This results in improved imaging of the anterior segment of the eye to be examined by means of retroillumination from the retina, which does not require additional image processing to eliminate reflections.

As a variant, the means for modulating a portion of the light beam emitted by the illumination source suppresses a portion of the light beam and allows the emission of a light beam coming from a light source having a different wavelength in this extinguished region, making it possible to create differently illuminated regions of the eye, so as to create a so-called fixation beam to facilitate the examination. The imaging means then comprises means for blocking said fixation light beam reflected by the eye, such as a spectral filter, the spectral filter enabling the reflected and backscattered fixation light beam to be blocked as a function of its wavelength.

The device advantageously allows an image of the anterior segment to be captured while offering an image in which parasitic reflections intrinsic to the system and also possible secondary reflections of the corneal surface and of the natural or artificial crystalline lens are eliminated.

A device according to the invention thus makes it possible to diagnose and monitor common diseases such as, for example, Fuchs' corneal endothelial dystrophy, also called cornea guttata, at its early stage, which affects 4 to 10% of adults over age 40, or secondary cataract (opacification appearing behind a cataract surgery implant) which affects about 20% of patients. The term “cornea guttata” designates the presence of anomalies of the posterior face of the cornea in the form of microscopic “droplets” visible with the retroillumination device according to the invention, these droplets most often being the sign of the onset of Fuchs' corneal endothelial dystrophy.

The device according to the invention can advantageously be used to reliably image the cataract in order to evaluate its density and make the diagnosis objective.

The device according to the invention makes it possible to analyze the image (quantification of certain characteristic elements of the pathologies targeted) of these images in retroillumination in order to diagnose the severity of certain pathologies and to follow their progression over time (speed of worsening, aid in medical decision-making). Such a device according to the invention is much more advantageous than the current diagnostic devices which are based on a partial photo (excluding the areas of reflection from the analysis) or on taking at least two photos and then image reconstruc-tion.

Preferably, the illuminating means comprises reflecting means composed of a plane mirror, called reflection mirror, for projecting the illumination light beam toward the eye to be examined, which makes it possible to preserve the intensity of the light beam created. Consequently, the illumination path defined by these illuminating means is distinct from the imaging path defined by the imaging means. Since the two illumination and imaging paths do not have a common interface, their structure and implementation are therefore more modular. It is then possible to provide for the illuminating means and the imaging means to be in the form of two distinct modules. In particular, it is possible to offset the imaging path relative to the illumination path, the illumination and imaging optical axes being offset. In addition, it is thus possible to adapt these two paths to existing commercial slit lamps. It can also be provided that the optical axes of illumination and imaging are merged.

The offset between the illumination optical axis and the imaging optical axis can help to better highlight eye details. For example, it is possible to provide an angular offset comprised between 3° and 15°, and preferably between 5° and 10°, between the optical illumination axis and the optical imaging axis. Too large an angle runs the risk, especially with a wide field image, of uneven illumination, with a part of the image less well lit. Conversely, with an offset angle that is too small, or even zero, the structures in relief that can be seen in the images will have less contrast.

As a variant, a polarizing cube may be provided in the illuminating means constituting the polarizing means and which also replaces the reflecting means.

The object of the invention is also to propose a device for the examination of the anterior part of the eye by retroillumination, having means for illuminating the eye comprising a light source and means for imaging the anterior part of the eye, characterized in that the illuminating means comprises a light source of the light-emitting diode (LED) type, collimating means for forming a light beam such as an optical unit, a diaphragm, polarizing means, reflecting means for sending the light beam toward the eye of a patient, and means for shutting off part of the light beam formed constituting the means for modulating a region of the light beam.

Advantageously, the shutter means or shutter makes it possible to create a beam of light, one region of which is “cut”, thus extinguished by the shutter. The latter is preferably composed of an opaque shape interposed on the trajectory of the light beam, absorbing part of the light beam. Preferably, the shutter is inside the light beam, i.e., arranged so as to be surpassed on all sides by a non-extinguished part of the light beam.

The light emitted by the source is focused by an optical unit composed of a group of lenses such as a collimating lens as means for collimating the light emitted by the source and an afocal doublet formed by two lenses which focuses the beam on the diaphragm to optimize the power supplied, the diameter of the light beam thus formed being reduced. It is possible to envisage a collimating lens making it possible to output the beam with the correct diameter.

The shutter can then be placed in the path of the light beam, after the optical unit, upstream, in the center or downstream of the diaphragm.

The shutter, placed at the center of the light beam formed at the outlet of the optical unit, for example at the center of the diaphragm, prevents illumination of the central region of the eye which has a usual reflection, said shutter being in the plane of the diaphragm, both being conjugated with the imaging plane in front of the eye.

Preferably, the illuminating means of the device comprise at least one plane mirror interposed between the diaphragm and the reflecting means, reflecting the light beam leaving the diaphragm toward the reflecting means especially composed of a plane mirror, called a reflection mirror, for sending the light beam toward the eye of a patient. The two plane mirrors are inclined at 45° which advantageously reduces the overall size of the device.

The reflection mirror is preferably conjugated with the image focus of a lens, positioned between the two mirrors, in order to minimize the size of this reflection mirror, which maximizes the intensity of the received light beam sent to the patient.

The object of the invention is a device for the examination of the anterior part of a patient's eye by retroillumination, having means for illuminating the eye comprising a light source and means for imaging the anterior part of the eye, characterized in that the illuminating means comprises a light source of the light-emitting diode (LED) type, collimating means for forming a light beam, a diaphragm, polarizing means, reflecting means for sending the light beam toward the eye of a patient, and a mirror having at least one orifice, thus not returning part of the light beam emitted from the source and constituting the means for modulating a region of the light beam.

Preferably, the mirror is parabolic, thus also constituting the collimating means, which avoids the presence of an optical unit comprising collimating means as described above. It is also possible to provide a second mirror after the diaphragm, also parabolic. However, it is also possible to envisage that the mirrors are plane and in this configuration, the optical unit as described above is provided.

Advantageously, such a device thus has a structure that is simple and reliable with regard to the formation of the extinguished region of the light beam.

Moreover, preferably, the illuminating means comprises a second light source, positioned behind the first mirror and emitting a light beam through the orifice of said first mirror, the light beam coming from this light source being of a wavelength different from that of the light beam emitted by the first light source and forming a light beam called a fixation light beam.

Advantageously, the imaging means then comprise a spectral filter intended to extinguish the fixation light beam in the observed image.

The device according to this embodiment of the invention offers a reliable examination by making it possible to integrate a fixation light beam into the device in a simple way, thus allowing the patient to remain as immobile as possible during the examination, in order to have a better image resolution with less movement blur, and therefore obtaining better image quality.

Another object of the invention is to propose a device in which the means for modulating at least one region of the illumination light beam are composed of a spatial light modulator (SLM) and its control means which makes it possible to eliminate reflections specific to each patient in real time. Active, adaptive and rapid reflection suppression is thus obtained.

Such an SLM modulator has an optical function consisting of extinguishing one or more por-tions of the beam, such as turning pixels “on-off” by polarization, reflection, etc.

Thus, an SLM modulator may be a diffractive optical element modulating the phase of the light beam using liquid crystals. This system exploits the principle of anisotropy of liquid crystals, i.e., the modification of the index of liquid crystals, as a function of their spatial orientation. The orientation of the liquid crystals can be achieved by means of an electric field. Thus, by locally mod-ifying the index of the liquid crystals, it is possible to modify the wavefront of the light beam.

It is also possible to envisage that this SLM modulator is a digital micromirror device (DMD).

The SLM therefore allows dynamically shaping the wavefront of the light beam. This modulation allows shaping the projected beam in a dynamic and reconfigurable manner. It is thus possible to segment the reflections to obtain an image that is simple to process, the reflections being strongly distinguished from the background in terms of gray level.

This is lighting servo-controlled by computer processing of the imaging path. Its principle is as follows:
The image produced by the imaging path is analyzed by the computer;
The computer segments the areas of reflections;
This segmentation is transcribed into a spatial modulator to close a part of the emitted light beam and extinguish, in the lighting path, the areas responsible for reflections;
A new image of the modulator is projected in the imaging plane; the areas responsible for reflections are no longer illuminated. These steps are repeated in a loop to suppress the reflections in real time. Final image capture: Reflections are removed quickly while adapting to the patient's eye.

The illuminating means comprises a light source such as a light-emitting diode (LED). In this case, the examination device according to the invention can be coupled to a femtosecond laser (YAG) so as, in addition to diagnosis by means of the device according to the invention, to make it possible to perform an intervention by coupling it with a laser to carry out both diagnosis and treatment. Consequently, this medical procedure performed by hybrid devices combining laser slit lamps, for example, works much better when imaging is without reflection.

The invention, in its different embodiments, therefore offers a modular device of small size and moderate manufacturing cost. Moreover, this device, because of the improved image quality obtained, allows early diagnosis of pathologies such as guttata.

In addition, the device according to the invention can form part of an assembly which therefore combines this device for examining the anterior part of the eye by retroillumination of the anterior part of the eye, allowing a diagnosis by retroillumination and a device for treatment by YAG laser of secondary cataract or of floating body of the vitreous body or by femtosecond laser for cataracts.

### PRESENTATION OF THE FIGURES

The invention will now be described in more detail with reference to the drawing in which the figures represent:

FIG. 1 is a schematic representation according to a first embodiment of a device of the invention;

FIG. 2 is a schematic representation of the trajectory of the illumination and imaging light beams in the device according to FIG. 1;

FIG. 3 is a schematic cross-sectional representation of the illumination and imaging light beam at different positions in the trajectory according to FIG. 2;

FIG. 4 is a schematic representation according to a second embodiment of a device of the invention;

FIG. 5 is a schematic representation of the trajectory of the illumination and imaging light beams in the device according to FIG. 4;

FIG. 6 is a schematic cross-sectional representation of the illumination and imaging light beam at different positions in the trajectory according to FIG. 5;

FIG. 7 is a schematic representation according to a third embodiment of a device according to the invention;

FIG. 8 is a photograph of a cornea obtained by a device according to the invention;

FIG. 9 is a photograph of a crystalline lens obtained according to the invention.

### DETAILED DESCRIPTION

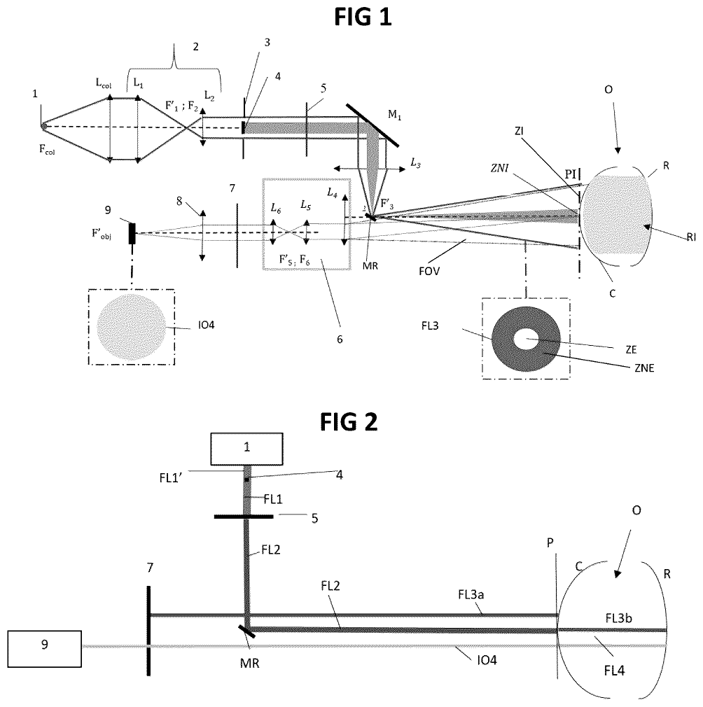

As can be seen in FIG. 1, an exemplary embodiment of a device according to the invention comprises a path for illuminating an eye O of a patient to be examined and an imaging path making it possible to observe this eye O and in particular to take images of the front segment of the eye.

The illumination path is defined by illuminating means comprising a light source 1 such as a light-emitting diode, (LED). The illuminating means is configured to emit a light beam FL1 in the direction of the eye O preferably covering the field of view FOV of the sensor of the imaging means at the surface of the eye O. Preferably, the light beam FL1 extends, in a plane of incidence to the eye O perpendicular to the light propagation, over an area having a diameter (largest dimension) of at least 4 mm, and preferably of at least 5 mm. The illumination of the large surface of the eye O allows more light to be brought into the eye O, making it possible to increase the backscattering and improve the acquired image.

The LED source 1 is chosen to present an emission spectrum whose peak is preferably close to 780 nm, corresponding to a light beam that is poorly absorbed by the eye and close enough to the visible so that the patient can fix the source 1 without being dazzled.

The intensity of the source 1 is modulated by control means in a conventional manner. Of course, the principle of reflection suppression can be applied to the whole spectrum of light; any other wavelength could be used.

The illuminating means then comprises an optical unit composed of a lens Lcol allowing the light emitted by the source 1 to be collimated toward an afocal doublet 2 formed by two lenses L1 and L2 whose foci F′1 and F2 coincide.

This afocal doublet 2 thus makes it possible to focus the light beam FL1 emitted by the source 1 on a diaphragm 3 in order to optimize the power supplied, the diameter of the beam FL1 thus being reduced.

The device according to the invention comprises means for modulating a region or part of the light beam FL1, making it possible to create a light beam on the eye of a patient by generating a so-called extinguished region in the light beam FL1 and thus create at least one non-illuminated region ZNI on the surface of the eye, while the non-extinguished regions of the light beam FL1 create at least one illuminated region ZI on the surface of the eye.

In the example shown in FIG. 1, the means for modulating the light beam is composed of a shutter 4, here positioned in the plane of the diaphragm 3. Preferably, the shutter 4 is composed of a form made of opaque material, preferably with a continuous shutter surface, such as, for example, an opaque disk. The shutter 4 may, for example, be held by support elements made of transparent material without incidence on the passing light beam FL1, for example in the plane of the diaphragm 3.

This light beam FL1 is then polarized by a polarizer 5 in the form of a light beam FL2.

In order to limit the size of the device, two plane mirrors M1 and MR are preferably provided. The mirror MR constitutes the means of reflecting the light beam toward the eye of the patient and the mirror M1, which may be optional, makes it possible to limit the size of the lighting means.

The mirror MR is associated with the image focus of a third lens L3 to minimize the size of the mirror MR, which maximizes the light beam FL2 received and sent to the patient's eye in the form of a light beam FL3, preferably divergent.

This light beam FL3 sent toward the patient passes, for a part of said light beam FL3, through the cornea C in the form of a light beam FL3b as far as the retina R on which this light beam FL3b is backscattered, depolarized in the form of a light beam FL4 which illuminates the imaging plane PI situated in front of the cornea C generating the retroillumination of the anterior segment and of the vitreous body of the eye. Part of the light beam FL3 is also reflected by the cornea C in the form of a light beam FL3a.

This reflected beam FL3a and the eye image IO4 then enter the imaging path. FIG. 2 shows all these light beams and their trajectories.

As can be seen, the shutter 4 interrupts the light beam FL1′ centrally. Consequently, it can be considered that the light beam FL1 at the outlet of the diaphragm 3 has a tubular section having an extinguished central region ZE and a non-extinguished peripheral region ZNE. The extinguished region prevents illumination of a region of the cornea C in correspondence with the shutter 4, as can be seen in FIG. 1. This non-directly illuminated region ZNI of cornea C no longer emits parasitic reflections preventing the examination of the anterior area of the eye by retroillumination. This non-illuminated region of the cornea ZNI corresponds to a region in which reflections systematically appear. In return, another region of the cornea C receives the light of the light beam not extinguished by the shutter 4 and forms a region ZI directly illuminated by the light beam. The imaging means is configured so that the image sensor comprises in its field of view FOV the non-illuminated region ZNI and at least one region ZI illuminated by the light beam on the surface of the eye.

The eye image IO4 is then captured by the imaging path defined by the imaging means and especially by the optical block 6 comprising a lens L4 making it possible to capture, in a manner offset from the optical axis of illumination, this image IO4 rejected to infinity by L4 and then to project this image IO4 towards infinity using two lenses L5 and L6 forming an afocal doublet, towards an optical sensor 9. This optical unit 6 corresponds to an optical head conventionally used in slit lamps. An optical head of this type makes it possible to alternate between five different magnifica-tions (×6 ×10 ×16 ×25 ×40).

At the outlet of this optical head 6, the image is rejected to infinity and one passes from one main optical axis to two optical axes as is provided for slit lamp eyepieces. Such an optical head 6 designed for imaging the anterior segment of the eye has the necessary optical qualities to image the cornea and the lens. This optical head 6 can be replaced by any optical unit or any optical head com-plying with the desired characteristics.

The eye image IO4 depolarized by the retina is then processed by polarizing means such as an analyzer (i.e. a polarizer 7) and then through the objective (lens L8) of an image capture apparatus, projected toward the focal point of the apparatus where the appropriate optical sensor 9 is located.

The polarizer 7 is linear, preferably screwed onto the objective 8 and is used with an axis crossed at 90° to the polarization axis of the light induced by the polarizer 5 of the illuminating means in order to eliminate the internal parasitic reflections of the optical system as well as a portion of the parasitic reflections, such as the portion of the light beam FL3 reflected by the cornea in the form of a light beam FL3a that has kept the same polarization as FL2.

In the embodiment shown, an optical sensor 9 is used which transforms photons into electrical charge by photoelectric effect, such as a complementary metal oxide semiconductor sensor (CMOS) or a charge-coupled device (CCD).

Preferably, a sensor 9 is chosen whose exposure time is preferably adjustable between 1 μs and 1 minute, which largely covers the exposure time in order to reduce motion blur and which has a resolution of 12 megapixels (3000×4096), i.e. a useful square area of 9 megapixels for imaging the cornea. A sensor of this type may be that offered under the commercial reference VCXU-123M. Any other type of sensor having appropriate characteristics may be used.

The optical sensor 9 is associated with software for recording images which, as has been seen, do not need image processing and are usable raw. However, it is possible to provide means for processing these images if necessary.

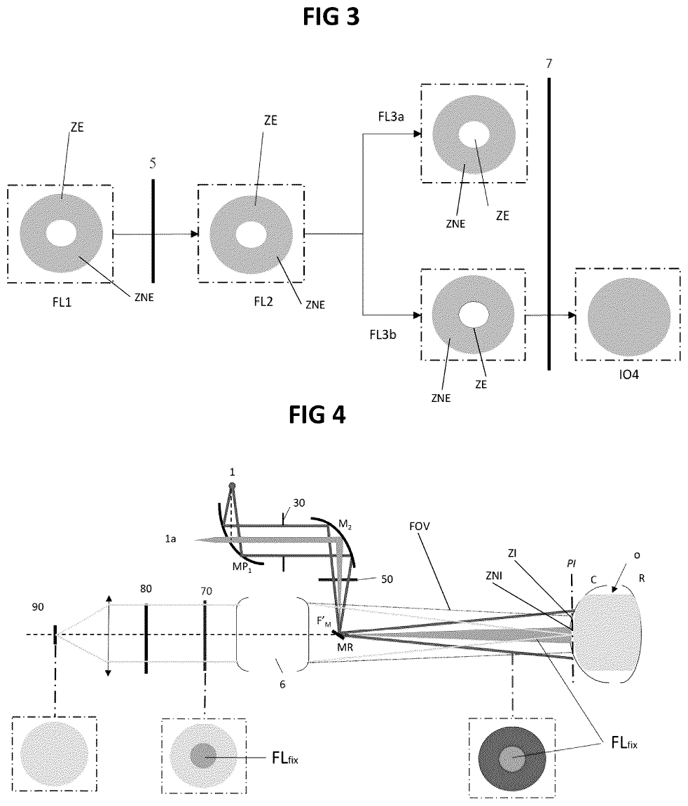

As can be seen in FIG. 3 which shows the light beams present at different stages of the trajectory between the source 1 and the optical sensor 9, the eye image IO4 is again a complete light image corresponding to the retroillumination by the retina R; the light beam FL3a is itself extinguished by the analyzer 7.

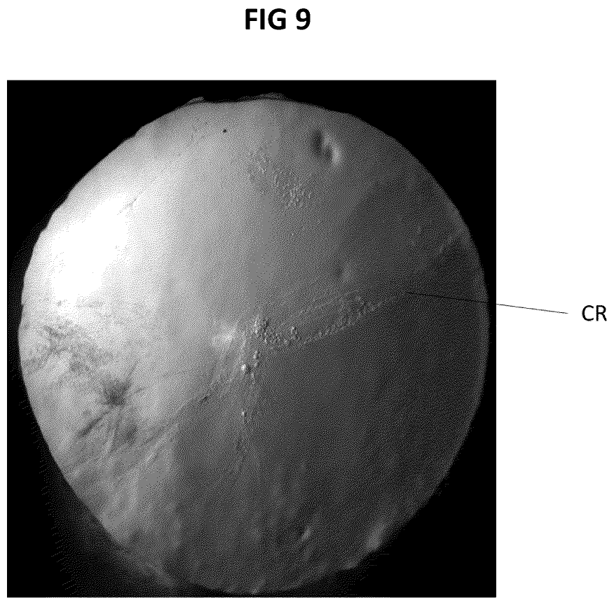

FIG. 8 shows a photograph of the anterior part, cornea C, of an eye examined by a device according to the invention, where it can be seen that there are no longer any reflections, while FIG. 9 represents a photograph of a crystalline lens CR.

In a second embodiment of the invention shown in FIGS. 4, 5 and 6, a variant of the illumination path is proposed which comprises two light sources emitting in different and distinct wave-lengths. For example, a first light source 1 emits a first light beam having a wavelength of 780 nm and a second light source 1a emits a second light beam having a wavelength of 550 nm. This second source 1a forms a different light beam called fixation FL_{fix}, enabling the patient, by fixing this light beam FL_{fix}, to remain immobile during the examination.

The illuminating means in this embodiment comprises a mirror MP1, preferably parabolic, which has a central hole which does not reflect the beam FL1 and forms within this beam F1 an extinguished region ZE and reflects a portion of the light forming the non-extinguished region (ZNE of the light beam. The parabolic mirror MP1 constitutes the means for modulating a region of the light beam and also makes it possible to collimate the light beam F1. This mirror MP1 thus has a dual function: optical and modulation. This extinguished region of the light beam FL1 is occupied by the fixation light beam FL_{fix} emitted by the light source 1a.

Thus, the fixation light beam FL_{fix} emitted by the second source 1a passes through the central orifice of the mirror MP1, thus creating a light beam FL10 coming from the two sources 1 and 1a.

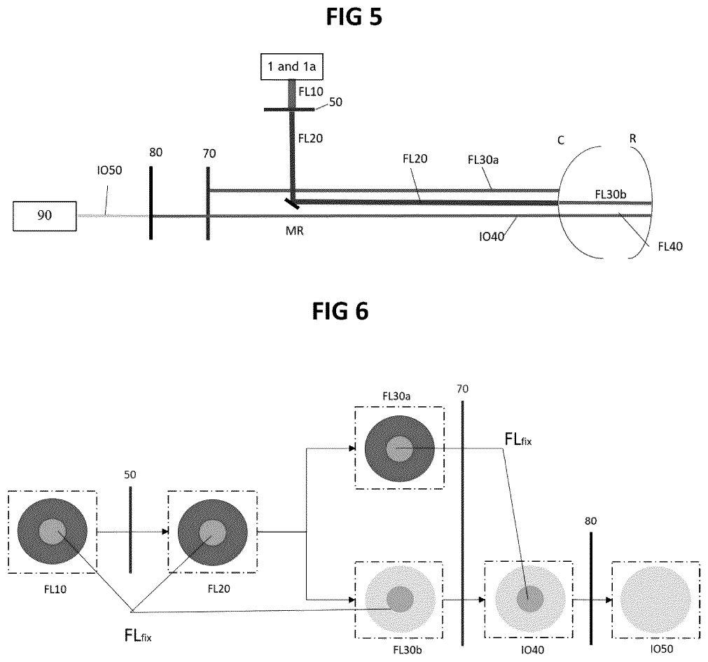

The light beam FL1 emitted by the source 1 is therefore reflected at 90° by a parabolic mirror MP1 in the form of a beam FL10 which integrates, in its extinguished region, the fixation light beam FL_{fix}. This beam FL10 passes through a diaphragm 30 to reach a second parabolic mirror MP2 on which it is reflected at 90° through a polarizer 50 in the form of a light beam FL20 toward a reflection plane mirror MR which then reflects it toward the eye to be examined. As can be seen in FIG. 4 and in FIG. 6, the light beam FL20 arriving on the cornea C is composed of the light beam FL1 with its extinguished region ZE and its non-extinguished region ZNE, and the fixation light beam FL_{fix}, which are merged and polarized. The parabolic mirror MP2 has the complementary function of focusing the beam (in addition to reflecting at) 90°.

The light beam FL20 is partly reflected by the eye in the form of a light beam FL30a while another part, FL30b, passes through the cornea and is backscattered and depolarized by the retina R in the form of a light beam FL40. This beam makes it possible to illuminate the anterior segment of the eye by retroillumination. The eye image IO40 is then captured by the imaging path, at an optical head 60 as described above, then of a polarizer/analyzer 70 as well as an appropriate spectral filter 80 to block the reflected light beam FL30a and let the IO40 pass while filtering the part coming from the fixation light beam. By filtering this “fixation” part of IO30, the fixation beam and reflections related to this central area are eliminated and an eye image IO50 is obtained without parasitic reflection.

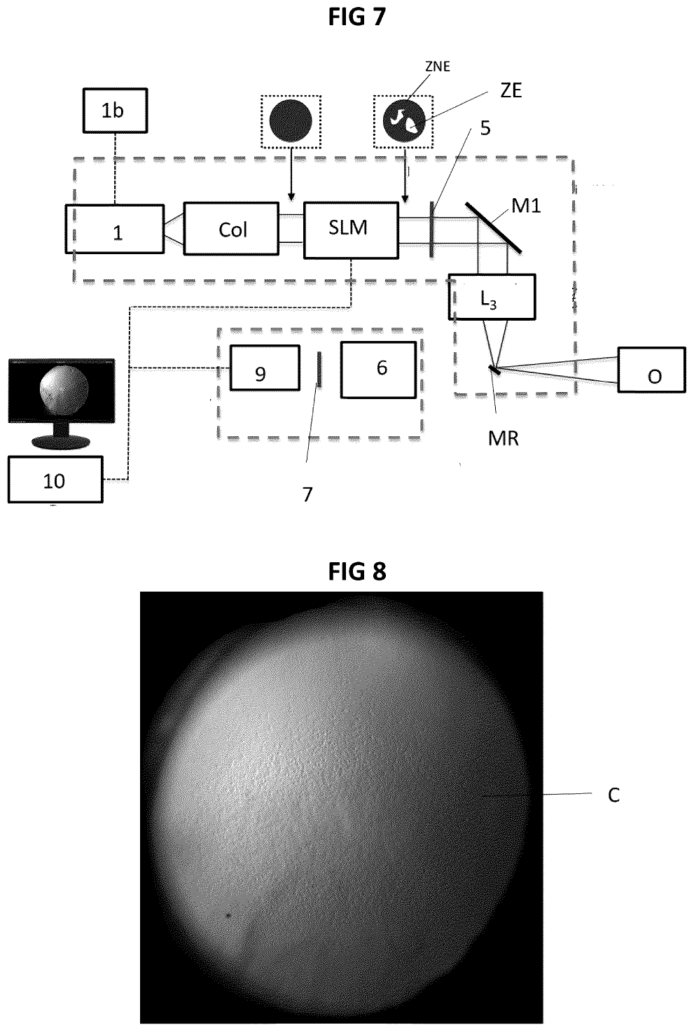

As can be seen in FIG. 7, a device according to the invention is described according to a third embodiment in which the illumination path comprises a light source 1, for example an LED light source, managed by control means 1b, a collimator Col, a spatial light modulator SLM, a polarizer 5, a first mirror M1, a lens L3, and then the mirror MR for reflecting the light beam toward the eye of the patient O.

The imaging path comprises an optical unit 6, an analyzer 7 and an optical sensor 9. A computer processing means (control software) is associated with this optical sensor 9 which makes it possible to eliminate in real time the specific reflections present on the surface of the image taken, for each patient. Adaptive and rapid reflection suppression is thus obtained. The computer processing means then makes it possible to control the illuminating means, the illumination being controlled by the computer processing of the imaging path.

The principle is as follows: The image produced by the imaging path is analyzed by a computer 10 which segments the regions of reflections detected and this segmentation is transcribed on the spatial modulator SLM to extinguish, in the lighting path, the regions responsible for reflections. A new image of the modulator is projected into the imaging plane; the regions responsible for reflections are no longer illuminated ZNI while the other directly illuminated regions ZI remain, these steps being repeated in a loop to remove reflections in real time. Reflections are therefore all removed quickly while adapting to the patient's eye, by means of the extinction of regions of the light beam.

In the various embodiments described, the modulating means is configured to extinguish at least one region ZE of the light beam FL1 emitted from the light source 1 and to thus create at least one non-illuminated region ZNI on the surface of the eye O. The light beam FL1 then comprises at least one extinguished region ZE and at least one non-extinguished region ZNE, the non-extinguished region ZNE creating an illuminated region ZI on the surface of the eye O.

The imaging means is configured so that the image sensor comprises in its field of view FOV the non-illuminated region ZNI and at least one region ZI illuminated by the light beam on the surface of the eye O. The field of view FOV is the solid angle through which the sensor is sensitive to light coming from the eye O, through the various components (lenses, etc.) of the imaging path.

Thus, the image acquired by the sensor of the imaging means represents both tissues corresponding to the non-illuminated region ZNI and tissues corresponding to the non-illuminated region ZNI. For example, in the case of the use of a central shutter 4, the image can show the center of the optical imaging axis corresponding to a non-illuminated region ZNI, but also parts of the anterior segment of the eye O that are eccentric, and corresponding to the illuminated region ZI surrounding the non-illuminated region ZNI on cornea C.

Since the field of view FOV includes a region ZI illuminated by the light beam, the use of an analyzer is all the more useful to eliminate secondary reflections related to the part reflected by the cornea.

Preferably, the imaging means is configured to present a field of view FOV covering an area of diameter greater than or equal to 4 mm on the eye O (e.g. in an imaging plane), and preferably greater than or equal to 5 mm. Such a field of view FOV allows imaging of the anterior segment of the eye O so as to make visible tissue that is not located in the immediate vicinity of the imaging optical axis. In addition, it is then possible to image a larger area of the anterior segment of the eye O without the need to repeat an image acquisition, and therefore the risk of movement of the eye O.

The modulating means is configured so that the extinguished regions ZE of the light beam represent less than 50% of the surface area of the non-extinguished regions ZNE in a section of the light beam, but preferably represent more than 5% of this surface region, and preferably more than 10%.

1. A device for examining an anterior part of an eye by retroillumination of the anterior part of the eye,
the device comprising means for illuminating the eye, the means for illuminating comprising a light source configured to emit a light beam,
the device comprising means for imaging the anterior part of the eye, the means for imaging comprising an optical sensor configured to capture an image of the anterior segment of the eye,
wherein the device further comprises means for modulating configured to extinguish at least one region of the light beam and to create at least one non-illuminated region on the surface of the eye.

2. The device according to claim 1, wherein the light source is of the light-emitting diode type, the means for illuminating comprising collimating means for forming the light beam, a diaphragm, polarizing means, means for reflecting the light beam toward the eye of a patient, and the means for modulating comprises means for shutting off part of the light beam.

3. The device according to claim 2, wherein the means for shutting off part of the light beam comprises an opaque disc.

4. The device according to claim 2, wherein the means for illuminating comprises at least one plane mirror interposed between the diaphragm and the means for reflecting.

5. The device according to claim 1, wherein the light source is of the light-emitting diode type, the means for illuminating comprising collimating means for forming the light beam, a diaphragm, polarizing means, the means for modulating comprises a mirror, the mirror having at least one orifice, the at least one orifice being configured to not reflect at least a part of the light beam.

6. The device according to claim 5, wherein the mirror is parabolic, the mirror being comprised in the collimating means.

7. The device according to claim 5, wherein the means for illuminating comprises a second light source, positioned behind the mirror, the light beam being a first light beam, the second light source being configured to emit a second light beam through the orifice of the mirror, a wavelength of the second light beam being different from a wavelength of the first light beam.

8. The device according to claim 7, wherein the means for imaging comprises a spectral filter intended to block a reflection of the second light beam.

9. The device according to claim 5, wherein the mirror is a first mirror, the means for illuminating comprises at least one second mirror interposed between the diaphragm and the means for reflecting.

10. The device according to claim 1, wherein the light source such is of a light-emitting diode type, the means for illuminating comprising collimating means for forming the light beam, polarizing means, means for reflecting the light beam toward the eye of a patient, means for spatial modulation of the light being provided to constitute means for modulating a region of the light beam.

11. The device according to claim 2 wherein the polarizing means and the means for reflecting comprises a polarizing cube.

12. The device according to claim 1, wherein the means for illuminating and the means for imaging are two distinct modules.

13. The device according to claim 1 wherein the means for imaging is configured so that a field of view of the optical sensor comprises the at least one non-illuminated region and at least one region illuminated by the light beam on the surface of the eye.

14. The device according to claim 1, wherein the modulation means is configured so that an extinguished region is surrounded by a non-extinguished region in the light beam.

15. The device according to claim 1, wherein the imaging means is configured to present a field of view covering an area of diameter greater than or equal to 4 mm on the eye.
